(19) United States
(12) Patent Application Publication
(10) Pub. No.: US 2003/0113932 A1
(43) Pub. Date: Jun. 19, 2003

(54) Hydrophobic ammonia sensing membrane

(76) Inventors: Shmuel Sternberg, Palatine, IL (US); Daniel Boggs, Lake Bluff, IL (US); Rosa Yeh, Libertyville, IL (US); Li Pan, Tampa, FL (US)

Correspondence Address: BAXTER HEALTHCARE CORPORATION, RENAL DIVISION, 1 BAXTER PARKWAY, DF3-3E, DEERFIELD, IL, 60015, US

(21) Appl. No.: 10/024,670

(22) Filed: Dec. 14, 2001

(51) Int. Cl. G01N 33/00; G01N 21/77

(52) U.S. Cl. 436/113; 436/163; 436/164; 436/165; 436/167; 436/169; 436/181; 422/55; 422/56; 422/83; 422/86

(57) ABSTRACT

A membrane capable of sensing gaseous phase ammonia is provided. The membrane includes a hydrophobic membrane layer that has a microporous structure and a pH sensitive dye embedded within the microporous structure such that the membrane is capable of calorimetrically sensing a change in ammonia with a high degree of sensitivity and responsiveness as applied to, for example, a change in ammonia dissolved in a dialysis solution during dialysis therapy.

### BACKGROUND OF THE INVENTION

The present invention relates generally to medical treatment. More specifically, the present invention relates to dialysis therapies.

Due to disease or insult or other causes, the renal system can fail. In renal failure of any cause, there are several physiological derangements. The balance of water, minerals (e.g., Na, K, Cl, Ca, P, Mg, SO₄) and the excretion of daily metabolic load of fixed hydrogen ions is no longer possible in renal failure. During renal failure, toxic end products of nitrogen metabolism (e.g., urea, creatinine, uric acid, and others) can accumulate in blood and tissues.

Dialysis processes have been devised for the separation of elements in a solution by diffusion across a semi-permeable membrane (diffusive solute transport) down a concentration gradient. Principally, dialysis comprises two methods: hemodialysis; and peritoneal dialysis.

Hemodialysis treatment removes uremic waste, toxins, and excess water directly from the patient's blood. The patient is connected to a hemodialysis machine and the patient's blood is pumped through the machine. Catheters are inserted into the patient's veins and arteries to connect the blood flow to and from the hemodialysis machine. Waste, toxins, and excess water are removed from the patient's blood and the blood is infused back into the patient. Hemodialysis treatments last several hours and are generally performed in a treatment center about three or four times per week.

Peritoneal dialysis utilizes a dialysis solution or dialysate, which is infused into a patient's peritoneal cavity. The dialysate contacts the patient's peritoneal membrane in the peritoneal cavity. Waste, toxins, and excess water pass from the patient's bloodstream through the peritoneal membrane and into the dialysate. The transfer of waste, toxins, and water from the bloodstream into the dialysate occurs due to diffusion and osmosis. The spent dialysate is drained from the patient's peritoneal cavity to remove the waste, toxins, and water from the patient.

There are various types of peritoneal dialysis, including continuous ambulatory peritoneal dialysis, and automated peritoneal dialysis. In general, continuous ambulatory peritoneal dialysis is a manual dialysis treatment where fresh dialysate fluid is delivered to and remains in the peritoneal cavity of the patient for a given dwell period subsequent to which the patient can connect an implanted catheter to a drain to allow spent dialysate fluid to drain from the peritoneal cavity. Automated peritoneal dialysis is similar to continuous ambulatory peritoneal dialysis in that the dialysis treatment includes a drain, fill, and dwell cycle. A dialysis machine automatically performs 3-4 cycles of peritoneal dialysis treatment, typically overnight while the patient sleeps. Compared to automated peritoneal dialysis, continuous flow peritoneal dialysis (CFPD) can significantly enhance small solute clearance as a result of the increased concentration gradient for diffusion and the increased peritoneal mass transfer coefficient.

With respect to continuous flow peritoneal dialysis, dialysate regeneration by a chemical cartridge can be utilized to reduce therapy volume as well as increase instrument portability. This is particularly useful for home based dialysis therapies. Existing dialysate regeneration cartridges based on urea catalysis produce ammonia in equilibrium with ammonium ions which is known to be toxic at high concentrations. A fixed capacity cationic exchanger can then be utilized to remove ammonium ions.

In this regard, the cartridge effluent must be continuously monitored for ammonia/ammonium concentrations to determine whether the capacity of the cartridge has been exceeded or a single point failure has occurred at the cationic exchanger. Previous clinical studies on chemically regenerated hemodialysate (e.g., REDY 2000) suggest a total amount of ammonia and ammonium ion detection threshold at 20 ppm which indicates a warning level and at 50 ppm which indicates to stop therapy. To this end, there exists a need to monitor the concentrations of ammonia and/or ammonium ions in the regenerated dialysate.

In general, a variety of different sensors and techniques are utilized to detect gaseous phase ammonia. For example, solid-state gas sensitive materials are capable of exhibiting a response, such as a thermal change, with respect to the presence of ammonia. These materials are typically employed in ionic selective electrodes. Ammonia sensors are also known which employ both a pH and ammonia sensing mechanism. These types of sensors are typically applied to the detection of ammonia gas within a mixture of gases.

Further, ammonia sensing technologies are known which employ the use of conventional membrane materials to separate the ammonia gas from the solution in which it is dissolved. Once separated, the ammonia gas can then be delivered to an ammonia sensor or sensing material. The ammonia sensing material can be a component of an ammonia sensing device which is separate and apart from the membrane material. In addition, it is known to add the sensing material to the membrane material in a conventional manner.

However, known ammonia sensing technologies may not provide an adequate level of sensitivity and responsiveness with respect to the detection of gaseous phase ammonia, particularly ammonia gas that is dissolved in dialysis solutions. The ability of an ammonia sensor to provide both sensitivity and responsiveness to a change in an amount of ammonia in the dialysis solution during dialysis therapy is necessary to ensure and facilitate adequate monitoring and/or controlling of dialysis therapy based on a detectable level of ammonia in the dialysis solution.

Accordingly, there exists a need to provide improved ammonia sensing membranes and methods of producing same which can be utilized to monitor and/or control solute toxin removals (e.g., urea) during dialysis therapy based on detectable amounts of gaseous phase ammonia dissolved in the dialysis solution.

### SUMMARY OF THE INVENTION

The present invention provides improved membranes capable of sensing a gas dissolved in solution, such as ammonia dissolved in dialysate solution. The ammonia sensing membranes of the present invention include a hydrophobic membrane that has a microporous structure and a pH sensitive dye embedded within the microporous structure of the membrane. In this regard, the ammonia sensing membrane is capable of selectively detecting gaseous phase ammonia as the pH sensitive dye which is embedded within a surface of the microporous membrane structure composed of strands is colorimetrically active in the presence of gaseous phase ammonia.

During dialysis therapy, for example, the ammonia sensing membrane of the present invention can effectively be applied to detect the presence of gaseous phase ammonia in a dialysis solution with both an enhanced sensitivity and responsiveness to a change in the amount of ammonia as the dialysis therapy progresses. As applied to continuous flow peritoneal dialysis, for example, the removal of toxins from the dialysate solution to produce regenerated or fresh dialysate for reuse can be effectively monitored and/or controlled based on the detectable amount of gaseous phase ammonia.

To this end, in an embodiment of the present invention a membrane capable of sensing a gas dissolved in a solution is provided. The membrane includes a hydrophobic membrane layer including a polymeric compound containing fluorine wherein the hydrophobic membrane layer has a porous structure; and a pH sensitive dye embedded within the porous structure of the hydrophobic membrane layer wherein the membrane is capable of calorimetrically sensing the gas.

In an embodiment, the solution is a dialysate solution.

In an embodiment, the porous structure of the hydrophobic membrane layer includes a pore size of about 9 microns or less.

In an embodiment, the pore size ranges from about 2.5 microns to about 1 micron.

In an embodiment, the polymeric compound containing fluorine is selected from the group consisting of polytetrafluoroethylene, polyvinylidene difluoride, acrylic-based fluorinated polymers, fluorinated ethylene propylene polymers, copolymers thereof and combinations thereof.

In an embodiment, the polymeric compound containing fluorine consists essentially of polyvinylidene difluoride.

In an embodiment, the pH sensitive dye is selected from the group consisting of bromophenol blue, bromothymol blue, phenol red, methyl orange, methyl yellow, 2,4-dinitrophenol, 2,6-dinitrophenol and mixtures thereof.

In another embodiment, an ammonia sensor capable of detecting gaseous phase ammonia dissolved in a solution is provided. The ammonia sensor includes a sensing membrane including a hydrophobic membrane layer composed of a fluorine-containing polymeric compound wherein the hydrophobic membrane layer includes a microporous structure having a surface defined by a plurality of strands within the microporous structure; and a pH sensitive dye embedded on the surface of the microporous structure of the hydrophobic membrane layer wherein the pH sensitive dye is capable of selectively reacting with the gaseous phase ammonia such that the gaseous phase ammonia is calorimetrically detected.

In an embodiment, the microporous structure of the hydrophobic membrane layer includes a pore size of about 2.5 microns or less.

In an embodiment, the ammonia sensing membrane is capable of detecting a change in an amount of the gaseous phase ammonia within at least about three seconds.

In an embodiment, the ammonia sensing membrane is capable of selectively detecting an increase in the amount of gaseous phase ammonia.

In an embodiment, the ammonia sensing membrane is capable of selectively detecting a decrease in the amount of gaseous phase ammonia.

In yet another embodiment, a method of producing a membrane capable of calorimetrically sensing ammonia dissolved in a solution is provided comprising the steps of providing a hydrophobic membrane material including a polymeric compound containing fluorine wherein the hydrophobic membrane material includes a microporous structure; providing a pH sensitive dye; and adding the pH sensitive dye within the microporous structure of the hydrophobic membrane material.

In an embodiment, the pH sensitive dye is added to the hydrophobic membrane material by casting.

In an embodiment, a casting solution of the hydrophobic membrane material and the pH sensitive dye is processed under acidic conditions to form the membrane.

In an embodiment, the acidic conditions include adding an acidic solution containing methanol to the casting solution during processing.

In an embodiment, the pH sensitive dye is added to the hydrophobic membrane material by dip coating.

In an embodiment, the hydrophobic membrane is immersed in an aqueous solvent solution containing the pH sensitive dye during dip coating.

In still yet another embodiment, a method of producing an ammonia sensing membrane is provided comprising the steps of providing a hydrophobic membrane and a pH sensitive dye; mixing the hydrophobic membrane and the pH sensitive dye into a casting solution; and processing the casting solution under acidic conditions to form the ammonia sensing membrane such that the pH sensitive dye is embedded within a microporous structure of the hydrophobic membrane.

In a further embodiment, a method of producing an ammonia sensing membrane is provided comprising the steps of providing an aqueous solvent solution containing a pH sensitive dye; providing a hydrophobic membrane having a microporous structure; and immersing the hydrophobic membrane in the aqueous solvent solution such that the pH sensitive dye is embedded within the microporous structure of the hydrophobic membrane.

In yet a further embodiment, a method of detecting ammonia dissolved in a dialysis solution is provided comprising the steps of providing an ammonia sensing membrane capable of detecting the ammonia wherein the sensing membrane includes a hydrophobic membrane layer composed of a polymeric compound containing fluorine and a pH sensitive dye embedded within a porous structure of the hydrophobic membrane layer; and calorimetrically detecting a change in an amount of the ammonia with the ammonia sensing membrane during dialysis therapy.

In an embodiment, the change in the amount of ammonia is detectable within at least about three seconds.

In an embodiment, the ammonia sensing membrane is capable of detecting an increase in the ammonia dissolved in the dialysis solution.

In an embodiment, ammonia sensing membrane is capable of detecting an increase in the ammonia during dialysate regeneration.

In an embodiment, the ammonia sensing membrane is capable of detecting a decrease in the ammonia dissolved in the dialysis solution.

In an embodiment, the decrease in the ammonia is detectable to an amount of not less than about 0 ppm.

In still yet a further embodiment, a method of providing dialysis therapy is provided comprising the steps of providing an ammonia sensor including a sensing membrane having a hydrophobic membrane layer composed of a fluorine-containing polymeric compound and a pH sensitive dye embedded within a porous structure of the hydrophobic membrane layer wherein the ammonia sensor is capable of colorimetrically detecting ammonia dissolved in a dialysate solution; and selectively detecting an amount of the ammonia with the ammonia sensor.

In an embodiment, the step of monitoring and/or controlling toxin levels during dialysate regeneration based on the detectable amount of the ammonia dissolved in the dialysate solution is provided.

An advantage of the present invention is to provide improved membranes capable of sensing ammonia.

Another advantage of the present invention is to provide improved ammonia sensing membranes capable of calorimetrically detecting gaseous phase ammonia.

Yet another advantage of the present invention is to provide improved methods of producing membranes capable of detecting ammonia.

Yet still another advantage of the present invention is to provide improved methods of producing ammonia sensing membranes capable of detecting gaseous phase ammonia dissolved in dialysis solutions with enhanced sensitivity and responsiveness to a change in the amount of ammonia, particularly with respect to dialysate regeneration during continuous flow peritoneal dialysis therapy.

A further advantage of the present invention is to provide improved methods of providing dialysis therapy that employ ammonia sensing membranes with enhanced ammonia sensing capabilities.

Additional features and advantages of the present invention are described in, and will be apparent from, the following Detailed Description of the Invention and the Figures.

### BRIEF DESCRIPTION OF THE FIGURES

FIG. 1 graphically illustrates the correlation between ammonia levels detected by the sensing membranes of the present invention in comparison to levels determined by a chemical analyzer.

FIG. 2 graphically illustrates response times with respect to the detection of varying amounts of ammonia by a sensing membrane made in accordance with an embodiment of the present invention as compared to a commercially available membrane.

FIG. 3 graphically illustrates response times with respect to the detection of varying amounts of ammonia by a sensing membrane made in accordance with an embodiment of the present invention as compared to a commercially available membrane.

FIG. 4 graphically illustrates response times with respect to an increase in ammonia from 20 ppm to 30 ppm by a sensing membrane made in accordance with an embodiment of the present invention.

### DETAILED DESCRIPTION OF THE INVENTION

The present invention provides membranes capable of detecting a gas dissolved in solution and methods of producing and employing same. More specifically, the sensing membranes of the present invention include a membrane and a pH sensitive dye which is intimately embedded within a microporous structure of the membrane material. In this regard, the membranes of the present invention can selectively detect gaseous phase constituents, such as ammonia, oxygen, carbon dioxide, the like or combinations thereof, dissolved in solution.

As applied to dialysis therapy, the selective detection capabilities of the membranes of the present invention can allow the membranes to detect varying amounts of gaseous phase constituents, such as ammonia, in dialysis solutions with enhanced sensitivity and responsiveness to said changes. During continuous flow peritoneal dialysis, for example, the detection capabilities of the membranes of the present invention can be particularly applied to the detection of ammonia in regenerated dialysate. As previously discussed, regenerated or fresh dialysate can be produced by removing toxins, such as urea, from dialysate via a chemical cartridge (e.g., based on urea catalysis to convert urea to ammonia/ammonium ion) and an absorption process (e.g., cationic exchanger to remove ammonia/ammonium ion).

The regenerated dialysate can then be reused, rather than disposing of the spent dialysate. This can reduce therapy volume, as well as increase instrument portability, particularly as applied to home-based dialysis therapy. In this regard, the membranes of the present invention can be effectively utilized to monitor and/or control the amount of toxins or removal thereof in regenerated dialysate based on the detectable amount of ammonia in the dialysis solution.

The membranes of the present invention can include a variety of different and suitable material components and can be produced in a variety of suitable manners. In an embodiment, the membranes include a membrane material that is hydrophobic in nature (e.g., a hydrophobic membrane material). The hydrophobic membrane material can be composed of a variety of different and suitable materials. In an embodiment, the membrane material includes polypropylene, polytetrafluoroethylene (“PTFE”), polyvinylidene difluoride (“PVDF”), fluorinated ethylene propylene polymers (“FEP”), acrylic-based polymeric compounds, acrylic-based fluorinate polymers, copolymers thereof, combinations thereof and other suitable polymeric compounds.

As applied to medical treatments, such as dialysis therapy, the membrane material must be compatible with respect to typical sterilizing procedures, such as by gamma radiation. In an embodiment, the sterilizable membrane materials include polymeric compounds containing fluorine, such as PTFE, PVDF, FEP, acrylic-based fluorinate polymers, copolymers thereof, mixtures thereof and other suitable medically sterilizable polymeric compounds. In a preferred embodiment, the polymeric compound of the membrane is substantially composed of PVDF. As discussed below, Applicants have demonstrated that this type of compound can be desirably utilized to produce the membranes of the present invention such that the membranes, for example, exhibit fast and suitable detection response times with respect to changes in the amount of ammonia; are gamma irradiatable; sensitive to amounts of ammonia on order of parts per million (“ppm”) or below; and are insensitive to solution pH changes without the presence of ammonia.

In an embodiment, the membrane material has a microporous structure defined by its pore size. It should be appreciated that the membranes can be made to include any variety of different and suitable pore sizes. In an embodiment, the pore size is about 9.0 micrometers (e.g., microns) or less, preferably from about 3.5 microns to about 0.2 microns, more preferably about 2.5 microns or less. In an embodiment, the pore size of the membrane material can range from about 2.5 microns to about 1 micron, preferably from about 2.0 microns to about 1.6 microns.

The membranes of the present invention include a pH sensitive dye. In an embodiment, the pH sensitive dye is intimately embedded or bound within the porous structure of the membrane material such that a negligible amount, if any, dye leaches from the membrane material when the membranes are exposed to liquid. It is suggested that the dye is embedded on a surface of the microporous structure defined by membrane strands within the microporous structure. In this regard, the dye can adsorb to at least a portion of the surface of the microporous structure such that the dye remains intimately bound (e.g., embedded) within the microporous structure even as the membrane is subject to varying environmental conditions.

Further, the hydrophobic membrane material effectively acts as a liquid impermeable barrier such that gaseous ammonia dissolved in solution passes into the porous structure of the membrane by diffusion to selectively react with the pH sensitive dye. Applicants have surprisingly demonstrated that the detection capability of the membranes of the present invention are highly sensitive and responsive with respect to changes in the amount of ammonia in solution, such as a dialysis solution (e.g. regenerated dialysate), compared to sensing membranes made from commercially available membranes as discussed below. The pH sensitive dye of the present invention can include a variety of different and suitable materials including, for example, bromophenol blue, bromothymol blue, methyl yellow, methyl orange, 2,4-dinitrophenol, 2,6-dinitrophenol, phenol red, mixtures thereof and other suitable dye sensitive materials.

In an embodiment, the present invention provides a method for producing the ammonia sensing membranes of the present invention. In general, the method includes embedding or binding a pH sensitive dye within the porous structure, particularly to the surface strands of the microporous structure of a suitable membrane such that the ammonia sensing membrane is provided. Thus produced, the sensing membrane of present invention is capable of selectively detecting ammonia in a gas phase with high sensitivity and responsiveness to changes in the amount of ammonia, such as changes in levels of gaseous phase ammonia dissolved in dialysis solutions during dialysis therapy.

### Casting of Ammonia Sensing Membranes

In an embodiment, the present invention provides a method of producing the membranes by casting. During casting, the hydrophobic membrane and the pH sensitive dye are formed into a casting solution containing a suitable solvent. It should be appreciated that any suitable amounts of the hydrophobic membrane material and pH sensitive dye can be blended or mixed into the casting solution. In an embodiment, the casting solution includes at least about 0.1% by weight of the pH sensitive material in a solution containing about 14% to about 24% by weight of a hydrophobic membrane, preferably about 19% to about 21% by weight.

The casting solution is then poured onto a casting substrate, such as a mesh material, or glass substrate, and further processed by immersing the casting solution in an acidic solution under suitable conditions such that a precipitate is formed. The precipitate is then further washed and dried under suitable conditions to form the sensing membrane such that the pH sensitive dye is embedded within a microporous structure of the membrane material. In an embodiment, the processed sensing membrane can be dried at temperatures ranging from about room temperature to about 100° C., preferably at about 60° C.

The casting procedure can include a variety of different and suitable process conditions and procedures. In an embodiment, the acidic solution used during precipitation contains a suitable amount methanol, including about 50% to about 100% by weight of methanol, preferably about 90% or more by weight at a pH of about 3 to about 4, preferably about 3. An example of the casting procedure illustrative of an embodiment of the present invention is discussed below. It should be appreciated that the example of the casting procedure as discussed below is not intended to limit the scope of the present invention.

### Casting Example

A casting solution was prepared by blending a PVDF solution (e.g., hydrophobic membrane material) with bromophenol blue (pH sensitive dye). The casting solution included about 0.1% by weight of bromophenol blue in a solution containing about 20% by weight of PVDF. The PVDF solution was made with a suitable solvent including, for example, dimethyl acetamide, dimethyl formamide, triethyl phosphate, dimethyl sulfoxide or the like.

A methanol bath solution was prepared by mixing methanol with varying amounts of 0.1 N hydrochloric acid (“HCl”) to adjust the pH of the acid bath solution. The pH of the methanol bath solution ranged from about 3 to about 4. An acid washing solution was separately prepared with 0.1 N HCl having a pH of about 3.

A polyester support mesh passes through the casting solution continued in a V-shape dispensing device, sometime referred to as a V-box. The mesh exits and draws the casting solution through a slit on the V-box. The entire mesh structure becomes coated by and impregnated with the solution such that the casting solution is evenly spread on both sides of the mesh. The mesh substrate was a commercially available mesh material (HOLLYTEX 3257, 4.25 inches wide, 1400 yard/roll). Once exited, the casting solution was immersed in the methanol bath for about 3 minutes to about 18 minutes such that a precipitate formed. The precipitate was subsequently dried in air at about room temperature to about 80° C.

It should be appreciated that the above casting procedure can be suitably modified. In this regard, the pH sensitive dye can be added at any suitable stage during the casting procedure. For example, the pH sensitive dye can be added to the acid bath solution and then to the casting solution to form the precipitate. The casting procedure can also include additional washing of the precipitate prior to drying. The washing can be conducted with the acid wash solution as discussed above or other suitable washing media including water. Further, the drying stage can be conducted at room temperature or conducted under suitably higher temperatures in order to decrease the drying time. In addition, a variety of other suitable substrates in place of the mesh material can be utilized, such a glass substrate or a metal substrate.

### Dip Coating and Ammonia Sensing Membranes

In an embodiment, the present invention provides a method of producing the ammonia sensing membranes of the present invention by dip coating. During dip coating, a preformed hydrophobic microporous membrane is immersed in an aqueous coating solution containing a pH sensitive dye and a solvent. In an embodiment, the solvent includes isopropyl alcohol, acetone, mixtures thereof and other suitable solvent materials. The coating solution can include any suitable amount of the pH sensitive dye and the solvent. In an embodiment, the coating solution includes at least about 0.05% by weight of the pH sensitive dye, preferably at least 0.2% by weight, in an aqueous solution containing about 10% to about 50%, preferably about 30% by volume of the solvent.

An example of the dip coating process illustrative of an embodiment of the present invention is discussed below. It should be appreciated that the example of the dip coating process is not intended to limit the scope of the present invention.

### Dip Coating Example

A dip coating solution was prepared by mixing about 0.2% by weight bromophenol blue into an aqueous solution containing about 30% by volume of isopropyl alcohol.

A hydrophobic microporous membrane was prepared that included about 19% to about 21% by weight of PVDF. The hydrophobic microporous membrane was immersed into the dip coating solution for about 2 to about 8 minutes and then air dried to form the ammonia sensing membrane. It should be appreciated that the dip coating process of the present invention can be modified in a variety of different and suitable ways.

### Experimental Tests

Applicants conducted a number of experiments to demonstrate the efficacy of the membranes of the present invention. The membranes that were tested were prepared by procedures in accordance with an embodiment of the present invention as described above.

Porous Microstructure

The porous microstructure of the membranes made in accordance with an embodiment of the present invention were characterized employing a scanning electron microscopy technique. The test results demonstrated that the membranes of the present invention have a microporous sponge type structure.

pH Effects

The membranes of the present invention were tested to determine the effects of pH on the membranes ability to detect ammonia. In a first experiment, the membranes made in accordance with an embodiment of the present invention were placed in an ammonium hydroxide solution, a 1N sodium hydroxide solution, a 0.01N sodium hydroxide solution and a sulfuric acid solution.

The membranes immersed in the ammonium hydroxide solution exhibited a color change indicative of the presence of ammonia. In contrast, the membranes displayed negligible color changes, if any at all, when immersed in each of the remaining test solutions. This demonstrates that changes in the pH have negligible, if any, effects on the calorimetric reactivity of the ammonia sensing membranes of the present invention without the presence of ammonia.

In a second test experiment, the effects of pH were evaluated with respect to an ammonia sensing membrane made in accordance with the present invention. The ammonia sensing membrane was made by dip coating a PVDF hydrophobic microporous membrane in a 0.2% solution of bromophenol blue as previously discussed. The test sensing membrane was divided into ten separate test membrane samples (Sample Nos. 1-10). Sample Nos. 1, 3, 5, 7 and 9 were immersed in a test peritoneal dialysis solution (“PD-4”) that contained 1.5% dextrose at a pH of 5, 6, 7, 8 and 9, respectively. No ammonia was present in the test solution. Sample Nos. 2, 4, 6, 8 and 10 were immersed in the PD-4 test solution in addition to 20 ppm of ammonium chloride at a pH of 5, 6, 7, 8 and 9, respectively.

During testing, Sample Nos. 1, 3, 5, 7, and 9 exhibited effectively no color change. This again demonstrates that a change in pH, without the presence of ammonia, has essentially no effect on the colorimetric activity of the ammonia sensing membranes of the present invention. On the other hand, with respect to Sample Nos. 2, 4, 6, 8 and 10, the intensity of the color of the test sensing membranes increased as the pH of the test solution increased. This demonstrates that at a higher pH level, the equilibrium between ammonia and ammonium ions is favorable to ammonia. Thus, with increased levels of ammonia in solution, the intensity of the color change in the sensing membranes increases.

Ammonia Selectivity

The sensing membranes made in accordance with the present invention were tested to demonstrate the detection capabilities with respect to the detection selectivity for ammonia in comparison to other materials. In this test example, the sensing membranes were tested to evaluate their detection selectivity with respect to ammonia in the presence of carbon dioxide. The test results indicated that the presence of carbon dioxide had negligible, if any, effects on the detection of ammonia. In this regard, the sensing membranes did not detect the carbon dioxide and, thus, exhibited an enhanced selectivity with respect to the detection of ammonia. The amount of ammonia detected by the sensing membranes of the present invention demonstrated an essentially linear correlation with respect to known amounts of ammonia as measured by Cobas.

Detection Accuracy

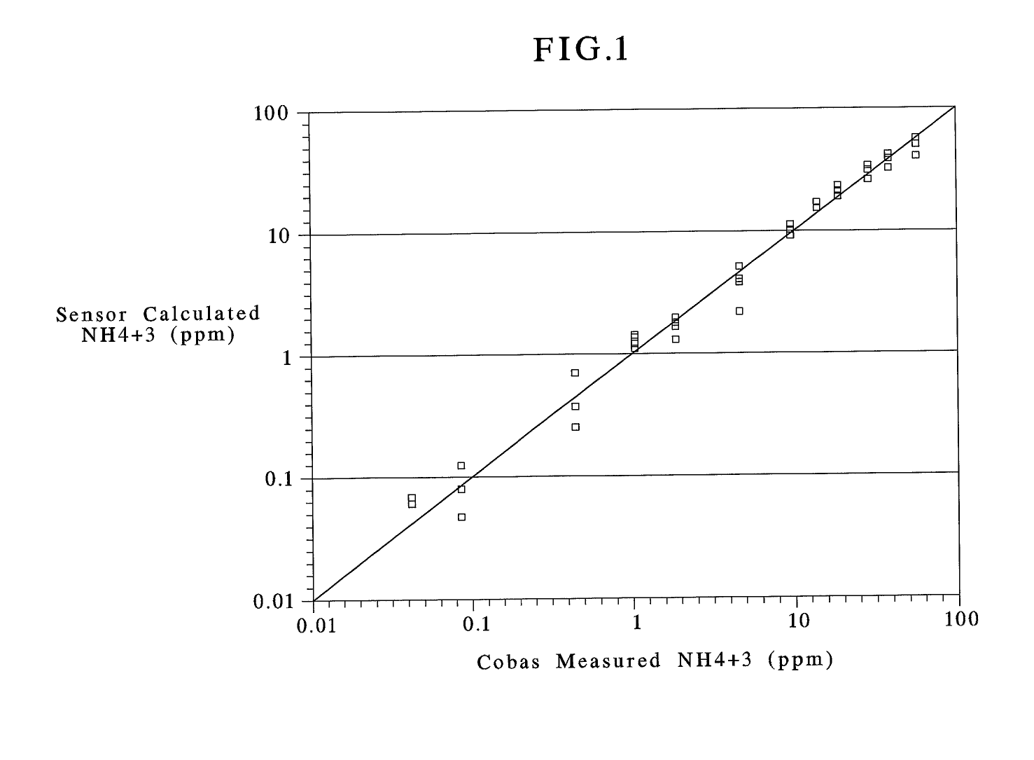

The membranes of the present invention were tested to demonstrate the accuracy and sensitivity of the ammonia detection capabilities of the present invention. The test results showed that the ammonia sensing membranes of the present invention accurately detected amounts of ammonia in a test solution ranging from about 0.01 ppm to about 100 ppm. This determination was based on a correlation (R²=0.8635) of the amount of ammonia detected by the membranes of the present invention versus the known amounts of ammonia in the test solutions ranging from about 0.1 ppm to about 100 ppm as measured by a chemical analyzer (e.g., Cobas Mira) as shown in FIG. 1.

Detection Response Times

The response times with respect to detecting ammonia were evaluated for the membranes of the present invention. In a first experiment, the effects of the hydrophobic membrane with respect to the detection response times were evaluated. In this experiment, four ammonia sensing membranes were prepared according to an embodiment of the present invention. The test sensing membranes varied with respect to the type of hydrophobic membrane. The hydrophobic membrane included three commercially available hydrophobic membranes and a PVDF microporous membrane made in accordance with an embodiment of the present invention. The response times with respect to the ammonia detection of the test membranes were evaluated under two separate test conditions.

Under a first test condition, each test sensing membrane was subjected to the PD-4 test solution as discussed above at pH 9. Under the second test condition, three of the test sensing membranes were subjected to ammonia gas in an amount of about 189 ppm. The test results are indicated below in the table:

| Hydrophobic | | Response Time | Response Time |
|---|---|---|---|
| Membrane | Manufacturer | PD-4 Test | Ammonia Gas |
| DURAPORE | MILIIPORE | Over 10 minutes | 2.5 minutes |
| CELGARD | HOECHST | 10 minutes | 1.5 minutes |
| | CELANESE | | |
| GORE | GORE THERAPY | Dye leached | N/A |
| PVDF | Present Invention | about 3 Seconds | 1.5 minutes |

As shown in the table above, the test sensing membrane made with a PVDF hydrophobic membrane material outperformed each of the test sensing membranes regarding the response time with respect to detecting ammonia in a peritoneal dialysis test solution. With respect to the second test condition, the test sensing membrane of the present invention performed as well as and better than the test sensing membrane made from commercially available hydrophobic membrane materials.

In a second test experiment, the response time with respect to detecting a change in an amount of ammonia from about 30 ppm to about 0 ppm in a test dialysate solution was evaluated. Two test ammonia sensing membranes were tested. A first sensing membrane was made from a PVDF hydrophobic membrane material in accordance with an embodiment of the present invention. A second sensing membrane was a commercially available sensing membrane from SEACHEM. The SEACHEM membrane was made from a polypropylene hydrophobic membrane material. The SEACHEM sensing membranes are typically used in aquarium applications.

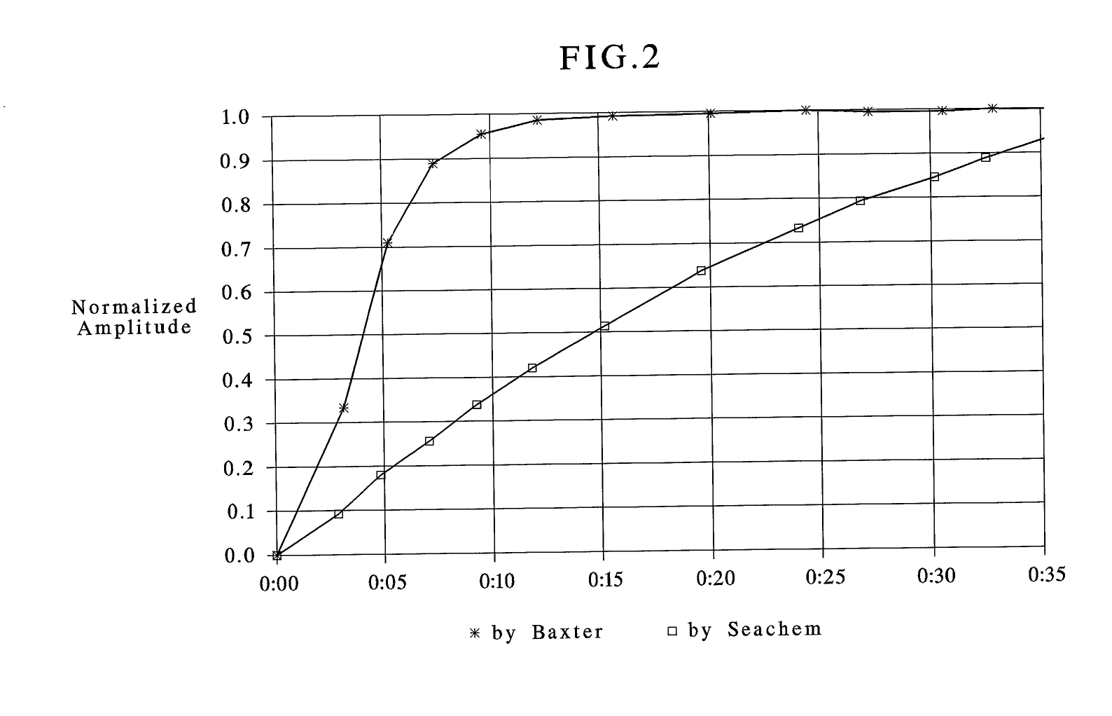

As shown in FIG. 2, the test ammonia sensing membrane of the present invention outperformed the commercially available sensing membrane. The detection response time with respect to the decrease in the amount of ammonia to about 0 ppm was about 12 seconds where a leveling off of the color change of the sensing membrane due to the decrease in the amount of ammonia began to occur. In contrast, the color change of the SEACHEM sensing membrane did not level off even after 35 seconds.

In a third test experiment, the response times with respect to detecting the changing amount of ammonia as the amount increases and decreases over a period of time were evaluated. The testing membranes included the SEACHEM sensing membrane as previously discussed and a sensing membrane made from PVDF in accordance with an embodiment of the present invention.

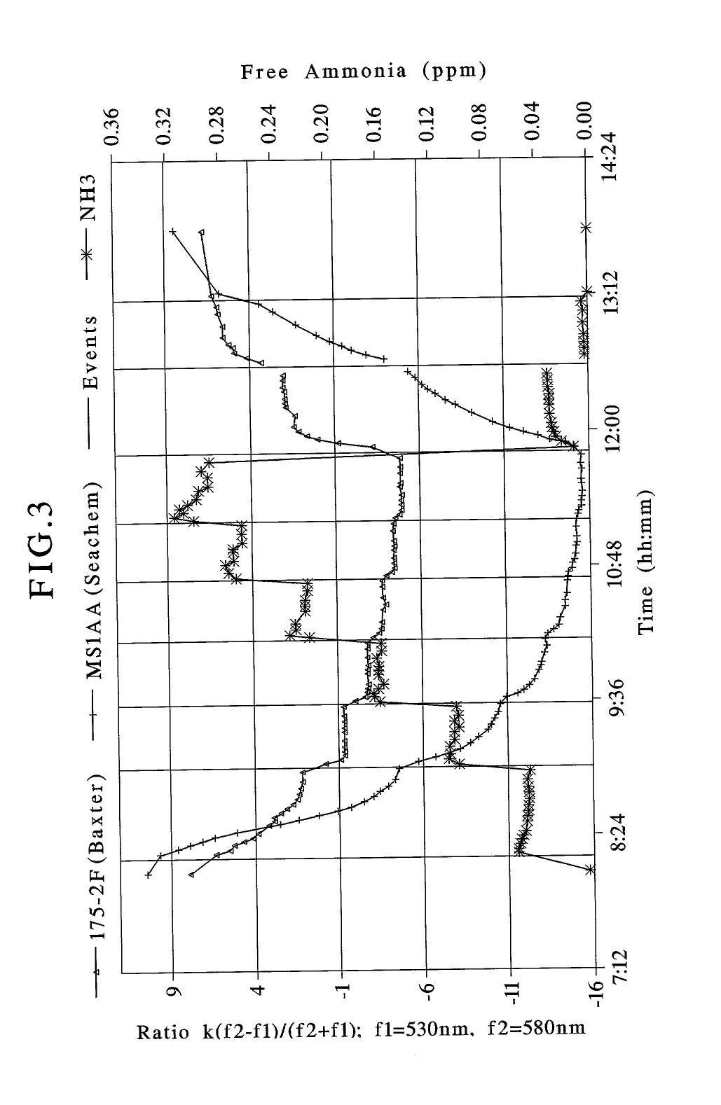

As shown in FIG. 3, the sensing membrane of the present invention detected the changes in the amount of ammonia with a greater level of sensitivity and responsiveness as compared to the commercially available sensing membrane.

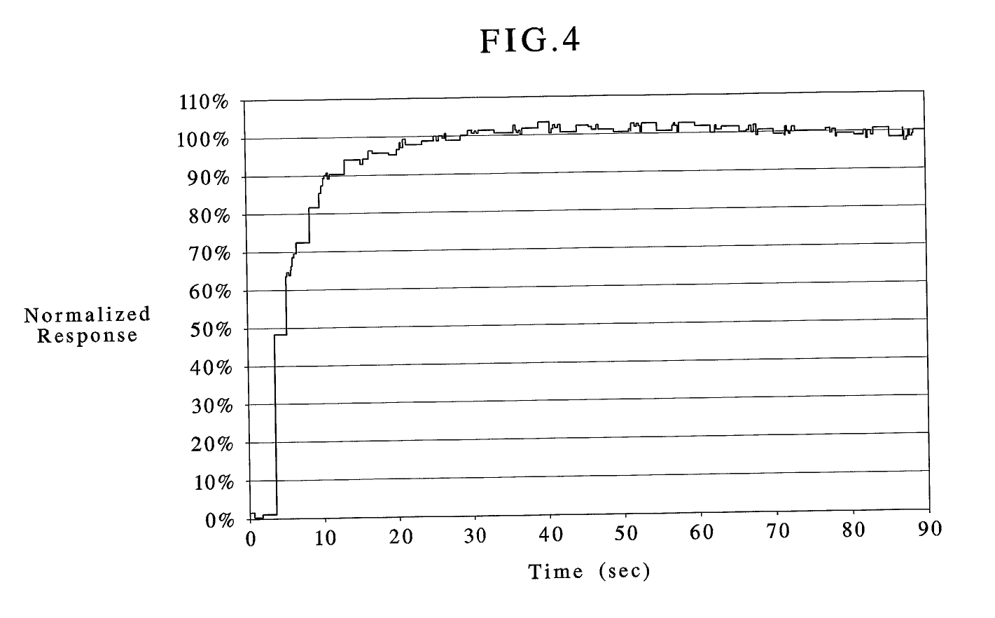

In a fourth experiment, a sensing membrane made in accordance with the present invention was subject to simulated clinical test conditions to evaluate its response time with respect to the detection of an increase in ammonia, namely an increase from about 20 ppm to about 30 ppm. As shown in FIG. 4, the sensing membrane detected the increase in the ammonia level in about 17 seconds (e.g., 16.47 seconds). This demonstrates the enhanced capabilities of the ammonia sensing membrane of the present invention to responsively detect a change in the level of ammonia.

As previously discussed, the ammonia sensing membranes of the present invention are capable of calorimetrically detecting the presence of ammonia in solution with a high degree of sensitivity, selectivity and responsiveness to changes in the amount of ammonia in solution. In an embodiment, the ammonia sensing membrane of the present invention can detect a change in an amount of ammonia within at least about three seconds. The response time can vary with respect to a number of conditions, such as pH as discussed above. In an embodiment, the pH ranges from about 5 to about 9, preferably about 9, in order to enhance the response time with respect to detecting the change in ammonia.

In an embodiment, the ammonia sensing membranes can detect an increase, a decrease, and combinations thereof of an amount of ammonia in solution over a period of time. In an embodiment, a decrease in ammonia can be detected to an amount of not less than about 0 ppm.

It should be appreciated that the ammonia sensing membranes can be utilized in a variety of different and suitable applications. For example, the ammonia sensing membranes of the present invention can be utilized during dialysis therapy. In an embodiment, the removal of toxins, such as urea, from fresh or regenerated dialysate during continuous flow peritoneal dialysis therapy can be monitored and/or controlled. This is based on the amount of ammonia calorimetrically detectable by the ammonia sensing membranes with a high degree of sensitivity and responsiveness.

The ammonia sensing membranes of the present invention can be adapted for use, such as during dialysis therapy, in a variety of different and suitable ways including, for example, as a total ammonia and ammonium sensor (TAAS) as disclosed in the patent application entitled “AMMONIA AND AMMONIUM SENSORS”, the entire disclosure of which is incorporated by reference into this disclosure and which is being filed in the United States Patent and Trademark Office simultaneously with the present application.

In an embodiment, the ammonia sensing membranes can be utilized as a stand alone sensor coupled to, for example, continuous flow regeneration dialysis system including, for example, a chemical cartridge and an absorption process during continuous flow peritoneal dialysis as previously discussed. In the alternative, the ammonia sensing membranes can be an integral component of a dialysis system.

In an embodiment, the ammonia sensing membrane of the present invention can be suitably adapted to process and communicate a signal associated with a calorimetric response to a detectable change in an amount of the ammonia. For example, the ammonia sensing membrane can be adapted to communicate with other components of a dialysis system, such as components of a continuous flow regeneration dialysis system, to monitor and/or control the removal of toxins from regenerated dialysate during dialysis therapy.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A membrane capable of sensing a gas dissolved in a solution comprising:
a hydrophobic membrane layer including a polymeric compound containing fluorine wherein the hydrophobic membrane layer has a porous structure; and
a pH sensitive dye embedded within the porous structure of the hydrophobic membrane layer wherein the membrane is capable of calorimetrically sensing the gas.

2. The membrane of claim 1 wherein the solution is a dialysate solution.

3. The membrane of claim 1 wherein the porous structure of the hydrophobic membrane layer includes a pore size of about 9 microns or less.

4. The membrane of claim 3 wherein the pore size ranges from about 2.5 microns to about 1 micron.

5. The membrane of claim 1 wherein the polymeric compound containing fluorine is selected from the group consisting of polytetrafluoroethylene, polyvinylidene difluoride, acrylic-based fluorinated polymers, fluorinated ethylene propylene polymers, copolymers thereof and combinations thereof.

6. The membrane of claim 1 wherein the polymeric compound containing fluorine consists essentially of polyvinylidene difluoride.

7. The membrane of claim 1 wherein the pH sensitive dye is selected from the group consisting of bromophenol blue, bromothymol blue, phenol red, methyl orange, methyl yellow, 2,4-dinitrophenol, 2,6-dinitrophenol and mixtures thereof.

8. An ammonia sensor capable of detecting gaseous phase ammonia dissolved in a solution comprising:
a sensing membrane including a hydrophobic membrane layer composed of a fluorine-containing polymeric compound wherein the hydrophobic membrane layer includes a microporous structure having a surface defined by a plurality of strands within the microporous structure; and
a pH sensitive dye embedded on the surface of the microporous structure of the hydrophobic membrane layer wherein the pH sensitive dye is capable of selectively reacting with the gaseous phase ammonia such that the gaseous phase ammonia is calorimetrically detected.

9. The ammonia sensor of claim 8 wherein the solution is a dialysate solution.

10. The ammonia sensor of claim 8 wherein the fluorine-containing polymeric compound consists essentially of polyvinylidene difluoride.

11. The ammonia sensor of claim 8 wherein the microporous structure of the hydrophobic membrane layer includes a pore size of about 2.5 microns or less.

12. The ammonia sensor of claim 8 wherein the ammonia sensing membrane is capable of detecting a change in an amount of the gaseous phase ammonia within at least about three seconds.

13. The ammonia sensor of claim 12 wherein the ammonia sensing membrane is capable of selectively detecting an increase in the amount of gaseous phase ammonia.

14. The ammonia sensor of claim 12 wherein the ammonia sensing membrane is capable of selectively detecting a decrease in the amount of gaseous phase ammonia.

15. A method of producing a membrane capable of calorimetrically sensing ammonia dissolved in a solution comprising the steps of:
providing a hydrophobic membrane material including a polymeric compound containing fluorine wherein the hydrophobic membrane material includes a microporous structure;
providing a pH sensitive dye; and
adding the pH sensitive dye within the microporous structure of the hydrophobic membrane material.

16. The method of claim 15 wherein the solution is a dialysate solution.

17. The method of claim 15 wherein the pH sensitive dye is added to the hydrophobic membrane material by casting.

18. The method of claim 17 wherein a casting solution of the hydrophobic membrane material and the pH sensitive dye is processed under acidic conditions to form the membrane.

19. The method of claim 18 wherein the acidic conditions include adding an acidic solution containing methanol to the casting solution during processing.

20. The method of claim 15 wherein the pH sensitive dye is added to the hydrophobic membrane material by dip coating.

21. The method of claim 20 wherein the hydrophobic membrane is immersed in an aqueous solvent solution containing the pH sensitive dye during dip coating.

22. The method of claim 15 wherein the polymeric compound containing fluorine is selected from the group consisting of polytetrafluoroethylene, polyvinylidene difluoride, acrylic-based fluorinate polymers, fluorinated ethylene propylene polymers, copolymers thereof and mixtures thereof.

23. The method of claim 15 wherein the pH sensitive dye is selected from the group consisting of methyl orange, methyl yellow, 2,4-dinitrophenol, 2,6-dinitrophenol, bromophenol blue, bromothymol blue, phenol red and mixtures thereof.

24. A method of producing an ammonia sensing membrane comprising the steps of:
providing a hydrophobic membrane and a pH sensitive dye;
mixing the hydrophobic membrane and the pH sensitive dye into a casting solution;
processing the casting solution under acidic conditions to form the ammonia sensing membrane such that the pH sensitive dye is embedded within a microporous structure of the hydrophobic membrane.

25. A method of producing an ammonia sensing membrane comprising the steps of:
providing an aqueous solvent solution containing a pH sensitive dye;
providing a hydrophobic membrane having a microporous structure; and
immersing the hydrophobic membrane in the aqueous solvent solution such that the pH sensitive dye is embedded within the microporous structure of the hydrophobic membrane.

26. A method of detecting ammonia dissolved in a dialysis solution comprising the steps of:
providing an ammonia sensing membrane capable of detecting the ammonia wherein the sensing membrane includes a hydrophobic membrane layer composed of a polymeric compound containing fluorine and a pH sensitive dye embedded within a porous structure of the hydrophobic membrane layer; and
calorimetrically detecting a change in an amount of the ammonia with the ammonia sensing membrane during dialysis therapy.

27. The method of claim 26 wherein the change in the amount of ammonia is detectable within at least about three seconds.

28. The method of claim 27 wherein the ammonia sensing membrane is capable of detecting an increase in the ammonia dissolved in the dialysis solution.

29. The method of claim 28 wherein the ammonia sensing membrane is capable of detecting an increase in the ammonia during dialysate regeneration.

30. The method of claim 27 wherein the ammonia sensing membrane is capable of detecting a decrease in the ammonia dissolved in the dialysis solution.

31. The method of claim 30 wherein the decrease in the ammonia is detectable to an amount of not less than about 0 ppm.

32. The method of claim 26 wherein the hydrophobic membrane layer consists essentially of polyvinylidene difluoride.

33. The method of claim 26 wherein the pH sensitive dye is selected from the group consisting of methyl orange, methyl yellow, 2,4-dinitrophenol, 2,6-dinitrophenol, bromophenol blue, bromothymol blue, phenol red and mixtures thereof.

34. A method of providing dialysis therapy comprising the steps of:
providing an ammonia sensor including a sensing membrane having a hydrophobic membrane layer composed of a fluorine-containing polymeric compound and a pH sensitive dye embedded within a porous structure of the hydrophobic membrane layer wherein the ammonia sensor is capable of calorimetrically detecting ammonia dissolved in a dialysate solution; and
selectively detecting an amount of the ammonia with the ammonia sensor.

35. The method of claim 34 further comprising the step of monitoring and/or controlling toxin levels during dialysate regeneration based on the detectable amount of the ammonia dissolved in the dialysate solution.

36. The method of claim 34 wherein the fluorine-containing polymeric compound is selected from the group consisting of polytetrafluoroethylene, polyvinylidene difluoride, acrylic copolymers, fluorinated ethylene propylene polymers and combinations thereof.

37. The method of claim 34 wherein the pH sensitive dye is selected from the group consisting of methyl orange, methyl yellow, 2,4-dinitrophenol, 2,6-dinotrophenol, bromophenol blue, bromothymol blue, phenol red and mixtures thereof.

38. The method of claim 34 wherein the porous structure of the hydrophobic membrane layer comprises a pore size of about 9 microns or less.
